(19) United States
(12) Patent Application Publication
(10) Pub. No.: US 2006/0286658 A1
(43) Pub. Date: Dec. 21, 2006

(54) METHOD FOR CONCENTRATED GROWTH OF A PARAFFINOPHILIC MICROORGANISM FOR BIOREMEDIATION AND AN ASSOCIATED APPARATUS

(76) Inventors: Justin Felder, Hermitage, PA (US); Mitchell S. Felder, Hermitage, PA (US)

Correspondence Address: ECKERT SEAMANS CHERIN & MELLOTT, 600 GRANT STREET, 44TH FLOOR, PITTSBURGH, PA, 15219, US

(21) Appl. No.: 11/425,170

(22) Filed: Jun. 20, 2006

Related U.S. Application Data
(60) Provisional application No. 60/692,476, filed on Jun. 21, 2005.

(51) Int. Cl. C12N 1/20; C12M 1/00

(52) U.S. Cl. 435/253.3; 435/287.9; 435/289.1; 435/262.5

(57) ABSTRACT

A method of growth of selected microorganism cultures by selectively baiting paraffinophilic microorganisms for concentrated growth is provided. The method includes providing a receptacle containing an aqueous solution and adjusting the solution to mimic the environmental conditions conducive to enhancing growth of the target microorganisms. The method then further includes inoculating the solution with a sample taken from an aqueous or terrestrial environmental site, or by seeding the aqueous solution with the paraffinophilic microorganism, chosen for its affinity for degrading organic waste or producing certain by-products and then placing in the receptacle one or more paraffin coated substrates to bait the paraffinophilic microorganism. An associated apparatus is also disclosed.

### CROSS REFERENCE TO RELATED APPLICATION

This application claims priority under 35 U.S.C. §119(e) to U.S. Provisional Patent Application Ser. No. 60/692,476, filed Jun. 21, 2005, entitled “Method for Selecting Paraffinophilic Microorganisms for Bioremediation.”

### FIELD OF THE INVENTION

The present invention relates generally to a method for augmenting growth of selected microorganism cultures by selectively baiting paraffinophilic microorganisms in a receptacle. More particularly, the invention relates to a method wherein material containing paraffinophilic microorganisms is placed in a receptacle having paraffin containing substrates in order to bait the paraffinophilic microorganisms contained in the material.

### BACKGROUND OF THE INVENTION

Federal and state governments regularly require that hazardous wastes, such as organic hazardous waste, and the sites on which they are located (contaminated sites) be treated to remove the hazardous waste. A widely used and accepted means for removing or degrading organic hazardous waste in aquatic and terrestrial sites is through bioremediation. Bioremediation can be defined as the process by which living microorganisms degrade or transform organic hazardous waste into less toxic or nontoxic substances. Most often, the microorganisms used for bioremediation are bacteria that have the capability of digesting organic hazardous waste which results in the breakdown of the hazardous waste into nontoxic components, such as carbon dioxide and water. It should be noted, however, that some bioremediation techniques require the use of other hazardous materials to degrade the hazardous material located on the contaminated site.

Conventional methods of remediation, apart from bioremediation, have included: excavating and transporting the contaminated materials to landfills, incineration, chemical treatment, air sparging, or pump-and-treat techniques. When compared to bioremediation, however, these alternatives carry the disadvantages of higher costs, greater health risks to workers, and the increasing difficulty of obtaining landfill availability.

By comparison, bioremediation offers the advantage of using nature in a comparatively much safer and more cost effective technique of remediation. Moreover, it offers an on-site, in situ technology that eliminates the need to dig up and transport organic hazardous waste. To this end, bioremediation technologies have been utilized to treat wastewater and contaminated soil at sites of contamination. For example, one bioremediation remedy is the addition of oxygen and nutrients to the contaminated site. The addition of the oxygen and the nutrients stimulates the growth of indigenous bacteria that can degrade the contaminated soil. In addition to the bioremediation techniques described above, another bioremediation technique is bioaugmentation. Bioaugmentation involves the addition of naturally occurring microorganisms to the contaminated site. Bioaugmentation typically involves culturing bacteria that have the ability to degrade the hazardous waste located in the contaminated site in a laboratory and adding the cultured bacteria to the contaminated site after a sufficient number of the bacteria have been produced. Once the cultured bacteria have been added to the contaminated site, oxygen, nutrients, or other materials that can enhance the degradation of the hazardous waste can be added to the contaminated site.

Certain paraffinophilic microorganisms belonging to the Pseudomonas genus, such as Pseudomonas putida and Pseudomonas fluorescens, are known to be efficient degraders of hazardous organic waste. For instance, it has been found that these microorganisms can degrade trichloroethylene, tetrachloroethylene (TCE), or Methyl Tertiary Butyl Ether (MTBE). However, the concentrations of these microorganisms in a site contaminated with hazardous waste might not be sufficient to efficiently degrade the hazardous waste. The Environmental Protection Agency has reported that there are naturally occurring microbes capable of degrading MTBE but they are “present natively in low numbers and take time to reach a sufficiently dense population to sustain MTBE degradation”. Accordingly, bioaugmentation methods have included the reconstitution of dried bacteria in a receptacle containing media solution and nutrients. However, there are a number of drawbacks to the bioaugmentation method that is described above. For instance, after reconstitution a majority of the bacteria might not be viable. Another drawback to the bioaugmenation method described above is the monetary expense associated with having to continually purchase bacteria to maintain a sufficient concentration of the microorganism at the contaminated site to degrade the hazardous waste.

### SUMMARY OF THE INVENTION

These needs and others are met by embodiments of the invention, which provide a method and apparatus for efficiently growing a high concentration of live paraffinophilic microorganisms for use in a bioremediation process.

In accordance with one embodiment of the invention, a method for the growth of paraffinophilic microorganisms suitable for bioremediation includes: adding an aqueous solution to a receptacle, inoculating the aqueous solution with a material containing paraffinophilic microorganisms to form a milieu, baiting the paraffinophilic microorganisms with one or more paraffin containing substrates, and growing the paraffinophilic microorganisms on the one or more paraffin containing substrates.

In accordance with another embodiment of the invention, an apparatus to facilitate growth of paraffinophilic microorganisms to biodegrade a waste material, includes a receptacle, one or more paraffin containing substrates within the receptacle, an aqueous solution contained within the receptacle, and an organic waste contained within the receptacle.

In accordance with one embodiment of the invention, the material containing paraffinophilic microorganisms may include material that originates from the contaminated site, a non-contaminated site, or from a microorganism culture lab or like source.

It is an object of the invention to provide a method and apparatus for growing paraffinophilic microorganisms in sufficient quantities for the bioremediation of a contaminated site.

It is another object of the invention to provide a method and apparatus for the growth of living paraffinophilic microorganisms for use in a bioremediation process.

It is another object of the invention to provide an efficient, effective, and economical way of growing and identifying a paraffinophilic microorganism that can be used in a bioremediation process.

### BRIEF DESCRIPTION OF DRAWINGS

A full understanding of the disclosed and claimed concept can be gained from the following Description when read in conjunction with the accompanying drawings in which:

FIG. 1 is a plan view of a paraffin substrate in a receptacle.

### DETAILED DESCRIPTION OF PREFERRED EMBODIMENTS

As used herein, the term “microorganism” and variations thereof refers to an organism of microscopic size including, but not limited to, bacteria, molds, and fungi.

As used herein, the term “paraffinophilic” and variations thereof refers to a microorganism that can employ paraffin as a source of carbon. An example of a paraffinophilic microorganism is that of the genus Pseudomonas, such as Pseudomonas putida.

As used herein, the term “organic waste” and variations thereof refers broadly to one or more carbon based compounds that are undesired. Organic waste generally can be consumed by bacteria and other small organisms. The term also includes “hazardous organic waste”, which refers to any organic substance or material that has been identified as hazardous or environmentally damaging by the Environmental Protection Agency or other authorized government agency including, but not limited to, refined petroleum products and natural as well synthetic hydrocarbons such as halogenated hydrocarbons (e.g. chlorinated hydrocarbons, trichloroethylene (TCE)).

As used herein, the term “contaminated site” and variations thereof refers to a location that has been contaminated with an organic waste. It is noted, however, that the term also includes the organic waste itself.

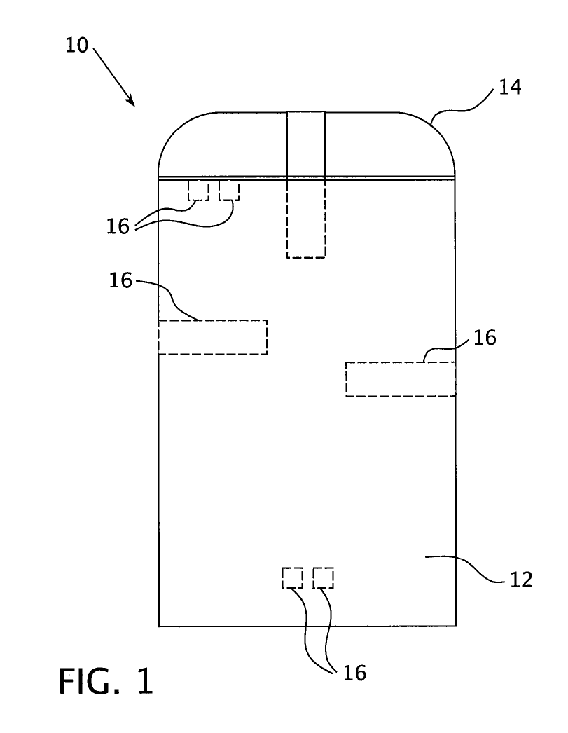

Referring to FIG. 1, a paraffinophilic microorganism growth apparatus 10 is shown. The microorganism growth apparatus 10 includes a receptacle 12 for containing an aqueous solution and a lid 14 that cover the receptacle 12. The lid 14 can either be removable from the receptacle 12 or movably mounted to the receptacle 12. Removing or moving the lid 14 provides access to the interior of the receptacle 12. The receptacle 12 can be of any size or shape that is suitable for holding an aqueous and waste material. For example, the receptacle 12 can be a test tube, drum, tank, bioreactor, or custom device. The receptacle can be manufactured from any material suitable for the purpose of holding an aqueous and waste material, including but not limited to, polyethylene, metals, plastics, glass or combinations thereof.

One or more paraffin containing substrates 16 is located within the interior of receptacle 12. These paraffin containing substrates 16 can either be secured to the receptacle 12 or the lid 14 or the paraffin containing substrates may be loosely placed within the interior of the receptacle 12. If the paraffin containing substrates 16 are loosely placed within the interior of the receptacle 12, the paraffin containing substrates 16 are preferably buoyant or are secured to a buoyant flotation device to ease removal of the paraffin containing substrates 16 from the receptacle 12. The paraffin containing substrates 16 can include, but shall not be limited to, substrates that are manufactured wholly or partially from paraffin and/or substrates that are coated or otherwise contain paraffin. The type of paraffin from which the paraffin containing substrates 16 are manufactured can be of any type of paraffin. For example, the paraffin can be a liquid paraffin. The shape of the paraffin containing substrates 16 can vary widely and may include, without limitation, blocks, pipes, rods, beads, slats, tubes, slides, screens, spheres, latticed substrates, and honeycombs. Regardless of the shape, the substrates 16 can be manufactured from materials that include, for example, glass, plastics, polyethylene, polystyrene, polypropylene, tetrafluoroethylene, silicones, paraffin or combinations thereof.

It should be noted that the apparatus 10 allows for using multiple substrates designed to maximize the surface area available for colonization of the microorganisms so that concentrated numbers of paraffinophjlic microorganisms may be grown and collected.

As stated above, certain paraffinophilic microorganisms belonging to the Pseudomonas genus, such as Pseudomonas putida and Pseudomonas fluorescens are known to be efficient degraders of organic waste or hazardous organic waste, such as trichloroethylene or tetrachloroethylene (TCE), or Methyl Tertiary Butyl Ether (MTBE). However, the concentrations of these microorganisms in a site contaminated with hazardous waste might not be sufficient to efficiently degrade the hazardous waste.

Accordingly, the invention provides a method for determining the presence of paraffinophilic microorganisms in paraffinophilic microorganism containing material and promoting concentrated growth of the paraffinophilic microorganisms on paraffin containing substrates 16 for the purpose of bioremediation a contaminated site. The present invention selectively grows target paraffinophilic microorganisms in sufficient concentrations (quantities) to perform effective bioremediation when introduced, in situ, into a contaminated site. For instance, the target microorganisms, once cultured, can be introduced into an organic waste infested aquifer or soil according to conventional standard practices. It will be appreciated that the method is adaptable to different bioremediation or degradation requirements, as the method can also be used as an enhancement to other conventional methods that are known in the art. For example, the cultured, selected paraffinophilic microorganisms that are grown using the disclosed invention may be introduced into an aquifer by using a pump to remove the aqueous solution from the receptacle 12. Once the paraffinophilic microorganism have been introduced into the contaminated aquifer, subsequent known process such as airsparging, adding nutrients, and recirculating groundwater in a contaminated aquifer may be utilized.

Referring to FIG. 1, the method provides adding an aqueous solution, such as distilled water, into the receptacle 12. The aqueous solution can be adjusted to mimic environmental conditions that are conducive to the growth of the paraffinophilic microorganisms by adjusting variables in the aqueous solutions such as pH, temperature, salinity, and/or oxygen levels using techniques that are well known in the art. Additionally, other nutrients conducive to growth that are well known in the art may be added to the receptacle as well. The aqueous solution is thereafter inoculated with a material containing one or more paraffinophilic microorganisms to grow in the milieu. The material can originate from a contaminated site, a non-contaminated site, or from a microbiology/microorganism culture lab or like source. The contaminated site from which the material can be extracted can include, for example, aqueous or terrestrial environmental sites, organic hazardous substances or food waste. If the material being extracted is originating from an organic waste, the organic waste may or may not be the organic waste on which the bioremediation process is to be performed on. The aqueous solution may further be inoculated with a paraffinophilic microorganism chosen for its affinity for degrading specific types of organic wastes or for producing certain by-products that may be collected using techniques well known in the art. Once the receptacle 12 has been inoculated with the material, one or more paraffin containing substrates 16 are placed in the receptacle 12 to bait paraffinophilic microorganism contained within the material. It should be noted, however, that the step of adjusting the environmental conditions of the aqueous solution can occur before or after the inoculation step and that the paraffin containing substrates 16 could also be placed within the interior of the receptacle 12 prior to the inoculation step. After the paraffin containing substrates 16 have been introduced into the receptacle 12, the inoculation step can be repeated as needed.

Cultures of the paraffinophilic microorganisms are generated on the paraffin containing substrates 16 because the paraffin in the paraffin containing substrates 16 baits the paraffinophilic microorganisms from the material. Once on the paraffin containing substrates 16, the paraffinophilic microorganisms use the paraffin that is within or on the paraffin containing substrates 16 as an energy source to multiply and thrive. The paraffin enhances the formation of microbial biofilms which adhere to the paraffin containing substrate's 16 surface. The formation of these biofilms provides survival advantages to the paraffinophilic microorganisms. For example, the paraffinophilic microorganisms on the paraffin containing substrate's 16 surface gain a level of protection against environmental threats. Furthermore, the paraffinophilic microorganisms having formed biofilms are able to obtain nutrients more efficiently since they are attached directly to the paraffin in the paraffin containing substrates 16. Accordingly, the paraffinophilic microorganisms expend less energy than if they were floating unattached in an aqueous liquid having optimal conditions for augmenting their growth.

Growth of microorganisms on the paraffin containing substrates 16 can be visually observed to determine the presence or absence of paraffinophilic microorganisms. Additionally, the paraffin containing substrates 16 can further be analyzed to determine the presence or absence of a specific paraffinophilic microorganism by utilizing more advanced testing techniques such as such as DNA hybridization or other techniques known in the art that can be tests for specific types of paraffinophilic microorganisms. Moreover, a culture of the paraffinophilic microorganisms on the paraffin containing substrate 16 can be tested for its effectiveness in degrading specific types of organic waste material (target material). One way this is accomplished is by introducing the target material that is to be degraded into the receptacle 16. The degradation of the target material would then be observed and monitored to determine the suitability of the baited paraffinophilic microorganism in degrading that specific type of organic waste. The results of this degradation testing would facilitate ascertaining whether the correct paraffinophilic microorganism is being cultured for a specific waste degradation need. Furthermore, this approach can also be used to train paraffinophilic microorganisms to degrade a specific type of organic waste, for example, a chlorinated hydrocarbon such as trichloroethylene, so that the paraffinophilic microorganism is released into the environment it will efficiently degrade the organic waste.

If subcultures of the paraffinophilic microorganisms are desired, the paraffin containing substrate 16 is removed from the receptacle 12 and the culture of paraffinophilic microorganisms that is on the paraffin containing substrate 16 is scrapped from the paraffin containing substrate 16 and subsequently transferred into a second, preferably larger, receptacle that contains one or more additional paraffin coated substrates 16 and an aqueous solution. Similar to the aqueous solution in the first receptacle, the aqueous solution in the second receptacle can be adjusted to mimic environmental conditions that are conducive to the growth of the paraffinophilic microorganisms by adjusting variables in the aqueous solutions such as pH, temperature, salinity, and/or oxygen levels using techniques that are well know in the art.

Periodically, the paraffinophilic microorganism biomass may be scraped off the paraffin containing substrates 16 into the aqueous solution in the receptacle 12 to obtain further growth in the aqueous liquid. After the paraffinophilic microorganism biomass has been scraped off, the paraffin containing substrates 16 may optionally remain in the aqueous solution to provide further adhesion and growth of select paraffinophilic microorganisms while additional paraffin containing substrates 16 may also be introduced into the aqueous solution to promote further growth of the microorganisms. This process may continue until concentrations of microorganisms achieved in the aqueous solution are in sufficient quantities for effective in situ bioremediation of a waste site to occur.

Once a sufficient concentration of paraffinophilic microorganisms has been achieved, a contaminated site can then be treated by inoculating the contaminated site with the cultured paraffinophilic microorganisms through the utilization of known bioaugmentation techniques. For example, the paraffinophilic microorganism seeded milieu can be pumped from the receptacle 12 and into the contaminated site or the paraffinophilic microorganisms can be added to the contaminated site after the paraffinophilic microorganisms have been isolated from the receptacle 12 by scraping the paraffinophilic microorganisms from the paraffin containing substrates 16. If additional paraffinophilic microorganisms are needed to inoculate the contaminated site, additional paraffin containing substrates 16 and additional aqueous solution are added to the receptacle 12 thereby allowing for the continuous generation of paraffinophilic microorganisms that can be used in the bioremediation process. By generating additional paraffinophilic microorganisms, bioremediation of a contaminated site can continue until applicable government standards are met.

In accordance with one embodiment of the invention, the method can also include stirring or shaking the receptacle 12 as needed for the purpose of encouraging biofilm growth of the paraffinophilic microorganisms. The act of stirring or shaking of the receptacle 12 can also cause the paraffinophilic microorganisms to detach (slough off) from the paraffin containing substrate 16 thereby allowing for dense growth of the detached paraffinophilic microorganisms in the milieu while allowing additional paraffinophilic microorganism colonies to grow on the paraffin containing substrates 16. Thus, a dense concentration of paraffinophilic microorganisms in both the milieu and on the paraffin containing substrates 16 may be optimized. Additionally, by stirring or shaking the receptacle 12 any nutrients or other additions to the milieu may more easily come into contact with the paraffinophilic microorganisms that are adhered to the paraffin containing substrate's 16 surface.

In accordance with one embodiment of the invention, one or more hollow tube shaped paraffin containing substrates 16 having an interior channel may be employed. The hollow tube shaped paraffin containing substrates 16 also have one or more apertures that are disposed on a surface of the hollow tube that open into the interior channel. A continuous supply of liquid paraffin may be fed into the interior channel of the hollow tube as needed, to maintain a steady source of growth materials for the long term maintenance of select paraffinophilic microorganisms colonizing the paraffin containing substrate 16. Accordingly, long term retention of microorganism communities capable of degrading hazardous organic waste is thereby facilitated.

In accordance with another embodiment of the invention, the method includes adding samples of an organic waste material to the receptacle 12 in order to condition the paraffinophilic microorganisms to use the organic waste material as a food or energy source.

Unlike other bioremediation techniques, the method disclosed in this invention does not necessitate using expensive and complicated equipment. Notably, it offers a non toxic approach to remediation, utilizing proven microorganism toxic waste degraders, such as those belonging to the Pseudomonas genus, which are not genetically altered and because they are naturally occurring, pose no threat to the environment. As a result, waste products may be eliminated by microorganism strains without releasing additional pollutants and without using environmentally unfriendly chemical agents. Additionally, the amount of paraffinophilic microorganisms released into the subsurface contamination environment for remediation return to the natural balance existing in the environment before treatment of the waste site, once the waste nutrient is degraded.

The method disclosed in this invention can also be used as a supplementary enhancement to other bioremediation techniques which reconstitute dried bacteria in a nutrient liquid media and require frequent purchases of dried bacteria products to maintain concentrated amounts for a waste degradation task. The present invention can complement other bioremediation systems by selectively selecting for and growing paraffinophilic microorganisms known for their degradation capabilities, by substantially ensuring that live organisms rather than dead organisms are present. While specific embodiments of the invention have been disclosed, it will be appreciated by those skilled in the art that various modifications and alterations to those details could be developed in light of the overall teachings of the disclosure. Accordingly, the particular arrangements disclosed are meant to be illustrative only and not limiting as to the scope of the invention which is to be given the full breadth of the appended claims and any and all equivalents thereof.

We claim:

1. A method for the growth of paraffinophilic microorganisms suitable for bioremediation of a contaminated site wherein the contaminated site is contaminated with an organic waste, comprising the steps of:
adding an aqueous solution to a receptacle;
inoculating the aqueous solution with a material containing one or more paraffinophilic microorganisms to form a milieu;
baiting the one or more paraffinophilic microorganisms with one or more paraffin containing substrates, and
growing the one or more paraffinophilic microorganisms on the one or more paraffin containing substrates.

2. The method of claim 1, wherein the material originates from the contaminated site, a non-contaminated site, or from a microorganism culture lab or like source.

3. The method of claim 1, further comprising the step of adjusting the aqueous solution's pH, temperature, salinity or oxygen levels.

4. The method of claim 1, further including the step of removing the one or more paraffinophilic microorganisms from the receptacle and placing the removed one or more paraffinophilic microorganisms in a second receptacle for growth therein.

5. The method of claim 1, further comprising the step of repeating the inoculation step.

6. The method of claim 1, further comprising the step of bioremediating a contaminated site outside of the receptacle with the one or more paraffinophilic microorganisms.

7. The method of claim 1, further comprising the step of pumping the milieu from the receptacle and into the contaminated site.

8. The method of claim 6, wherein the contaminated site is an aquifer or a terrestrial environment.

9. The method of claim 1, wherein a shape of any individual substrate of the one or more paraffin containing substrates is selected from the groups consisting of pipes, rods, beads, slats, tubes, slides, screens, latticework, honeycombs.

10. The method of claim 1, wherein the receptacle is covered with a lid.

11. The method of claim 10, wherein at least one of the one or more paraffin containing substrates is affixed or anchored to the lid.

12. The method of claim 1, wherein at least one of the one or more paraffin containing substrates are buoyant.

13. The method of claim 1, wherein the organic waste is a halogenated hydrocarbon-containing organic waste.

14. The method of claim 13, wherein the halogenated hydrocarbon is a chlorinated hydrocarbon.

15. The method of claim 1, further comprising the step of adding an organic waste to the receptacle to condition the paraffinophilic microorganisms to degrade the second organic waste, so that when released into a contaminated site the paraffinophilic microorganisms effectively degrade the second organic waste.

16. The method of claim 1, further comprising the step of testing the paraffinophilic microorganism for efficiency in degrading specific types of the organic waste.

17. An apparatus to facilitate growth of paraffinophilic microorganisms to biodegrade an organic waste material, the apparatus comprises:
a receptacle;
an aqueous solution contained within the receptacle;
a material containing paraffinophilic microorganisms is contained in the receptacle and is in contact with the aqueous solution; and
one or more paraffin containing substrates positioned within the receptacle and in contact with the aqueous solution.

18. The apparatus of claim 17, further comprises a lid that covers the receptacle.

19. The apparatus of claim 18, wherein at least one of the one or more paraffin containing substrates is attached or anchored to the lid.

20. The apparatus of claim 17, further comprises means to adjust the aqueous solution's pH, temperature, salinity, and oxygen levels.

21. The apparatus of claim 17, wherein at least one of the one or more one or more paraffin containing substrates are buoyant.
